(19) United States
(12) Patent Application Publication
(10) Pub. No.: US 2025/0325596 A1
(43) Pub. Date: Oct. 23, 2025

(54) MEDICAMENT FOR TREATMENT OF TRAUMA

(71) Applicant: Vernon Patrick MANCER, Germiston (ZA)

(72) Inventor: Vernon Patrick MANCER, Germiston (ZA)

(21) Appl. No.: 18/963,708

(22) Filed: Nov. 28, 2024

(30) Foreign Application Priority Data
Nov. 28, 2023 (ZA) 2023/07509

(51) Int. Cl. A61K 35/618; A61K 9/06; A61K 47/06; A61P 17/02

(52) U.S. Cl. CPC A61K 35/618; A61K 9/06; A61K 47/06; A61P 17/02

(57) ABSTRACT

The use of saltwater pearl powder in the manufacture of a medicament for use for treating trauma and the improved effectiveness in oxygenating the saltwater pearl powder.

This application is based on South African Provisional Patent Application No. 2023/07509 filed on 28 Nov. 2023 by the same inventor.

This invention relates to a medicament for the treatment of trauma to the integumentary system (commonly referred to as the skin).

According to this invention there provided the use of saltwater pearl powder in the manufacture of a medicament for treating trauma.

The medicament is conveniently used treating wounds such as (i) burns, (ii) cuts, (iii) tears or (iv) abrasions to the skin.

The tears and abrasions such as occur when a person falls off a motorcycle or motor vehicle travelling at speed over tar or rough surface.

Cuts such as occur after surgery or accidents with sharp instruments.

Burns such as caused by heat or exposure to electrical discharge, friction, chemicals, and radiation, including sunburn.

For this purpose, the medicament preferably comprises the combination of salt water pearl powder with a suitable viscus carrier such as petroleum jelly. For faster results, the salt water pearl powder may be previously oxygenated for a fairly lengthy period of time until the powder absorbs the ozone until saturated, preferably for about an hour. The oxygenation is done by means of an ozone generator.

Preferably only a small amount of the pearl powder is mixed into the petroleum jelly conveniently less than 5% w/w and preferably about 1% w/w.

In an example which will be described more fully below, a young black girl suffered third degree burns due to boiling water passing over her back and leg. She also lost the pigment under the burn. She was treated with the mixture of oxygenated saltwater pearl powder and petroleum jelly immediately. This saltwater pearl powder infused into the petroleum jelly was reapplied periodically. After about six weeks with daily treatment of the saltwater pearl powder infused petroleum jelly the burn healed. Subsequently the skin pigment returned.

In another example which will also be described more fully below, a man fell off a motorcycle travelling at speed over a tar surface. His hand engaged the surface which tore and abraded the skin on his hand. The skin was cleaned and then shortly thereafter treated by the medicament comprising oxygenated pearl powder and petroleum jelly and was completely cured in a short time.

For wounds such as burns and skin tears or abrasions the combination of oxygenated pearl powder and petroleum jelly and spread thickly over sterile gauze. The gauze is then applied to the wounds i.e. the burns and skin tears and cuts or abrasions.

An example of the medicament of the invention will now be described by way of example together with examples describing the use of the medicament will be described with reference to the pictures.

### A BRIEF DESCRIPTION OF THE PICTURES

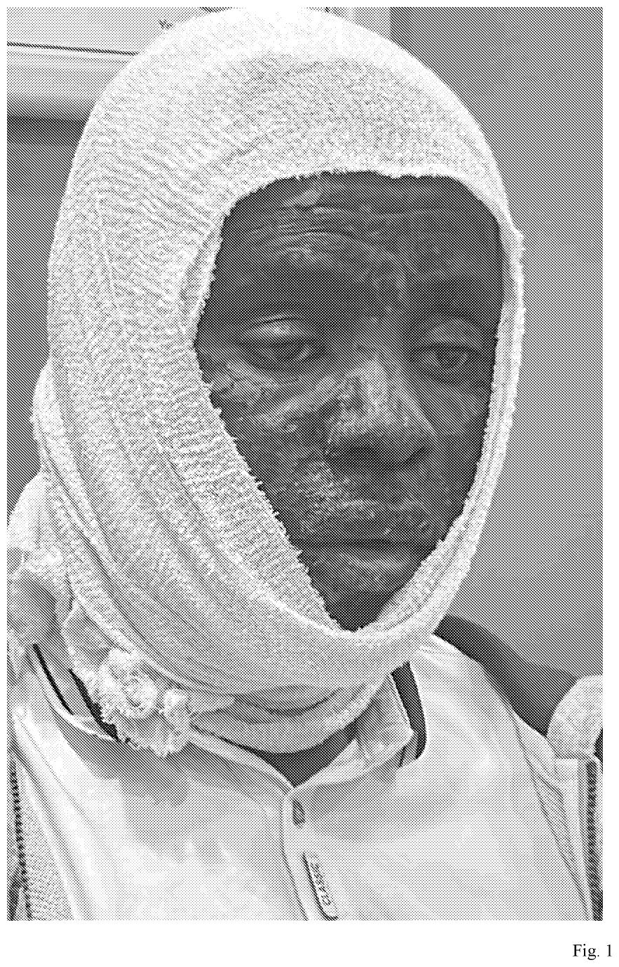

FIG. 1 shows a picture of a man who had burnt his face during a cooking oil accident in a commercial kitchen on the day of the burn;

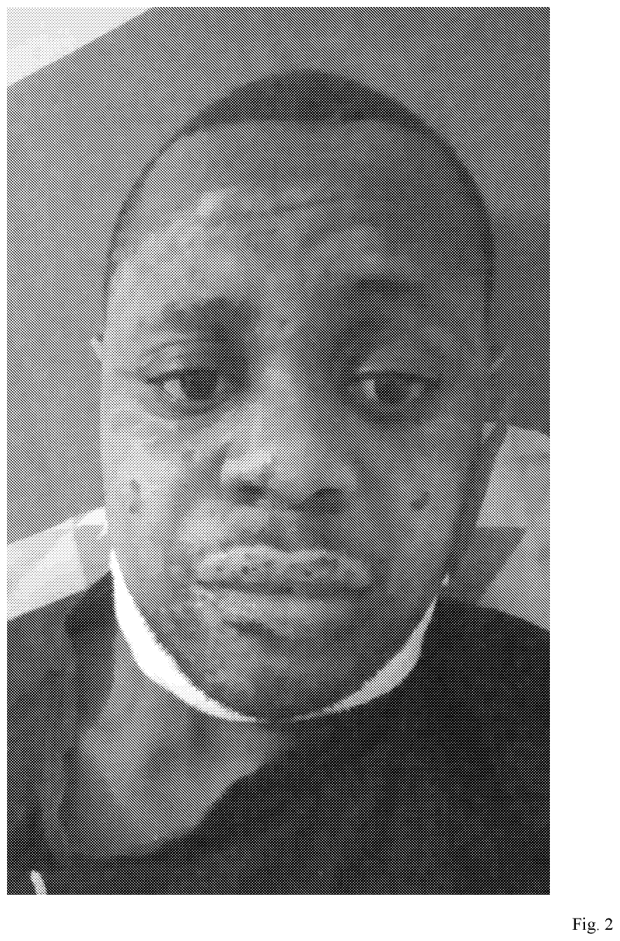

FIG. 2 shows the man who had burnt his face during a cooking oil accident in a commercial kitchen a week after the incident and the medicament of the invention had been used topically on his face;

FIG. 2 shows the man who had burnt his face during a cooking oil accident in a commercial kitchen five days after the incident and the medicament of the invention had been used topically on his face;

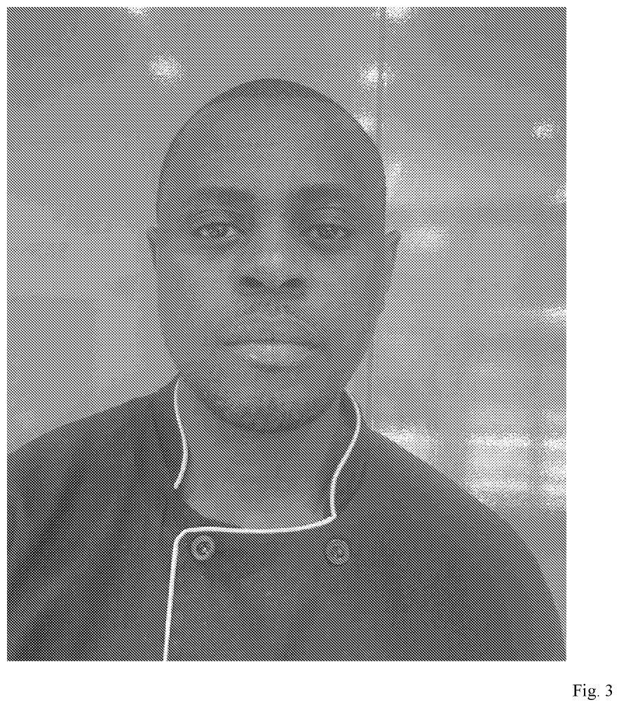

FIG. 3 shows the man who had burnt his face during a cooking oil accident in a commercial kitchen two months after the incident and the medicament of the invention had been used topically on his face;

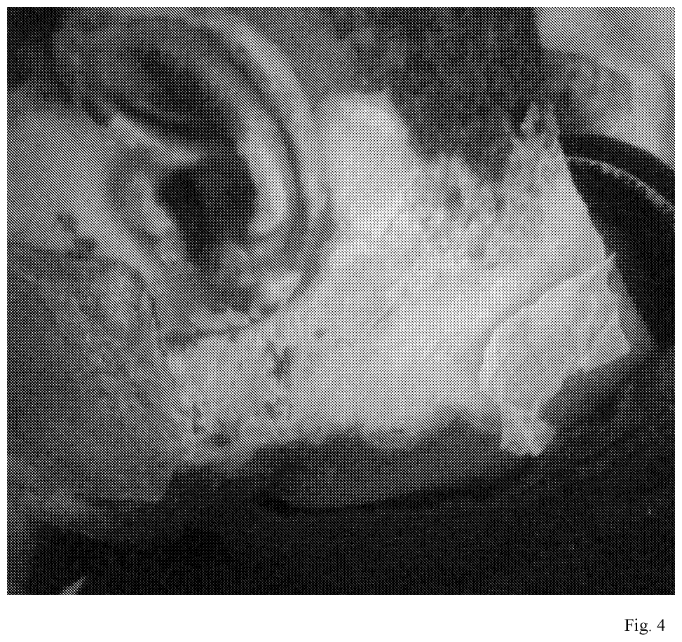

FIG. 4 is a view from the side of his neck and a portion of his face a week after the accident;

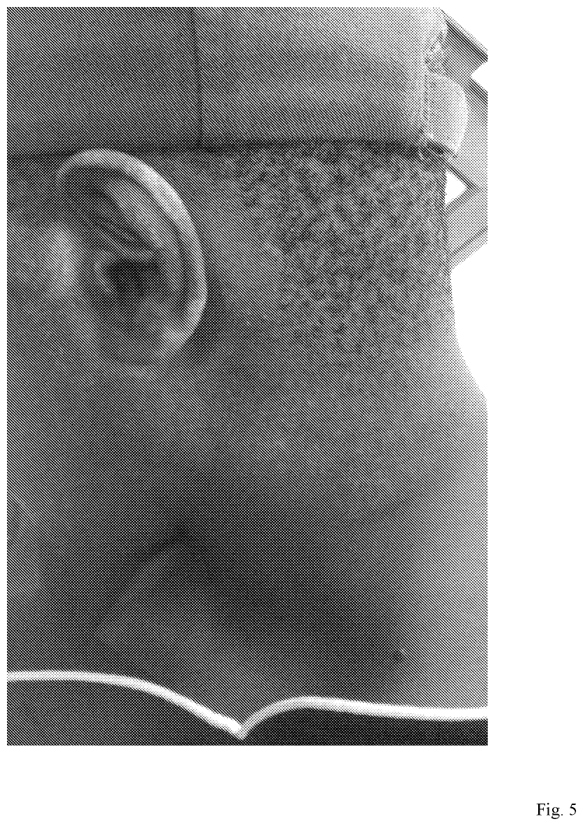

FIG. 5 is a picture from a similar angle two months after the incident and the medicament of the invention had been used topically on his face;

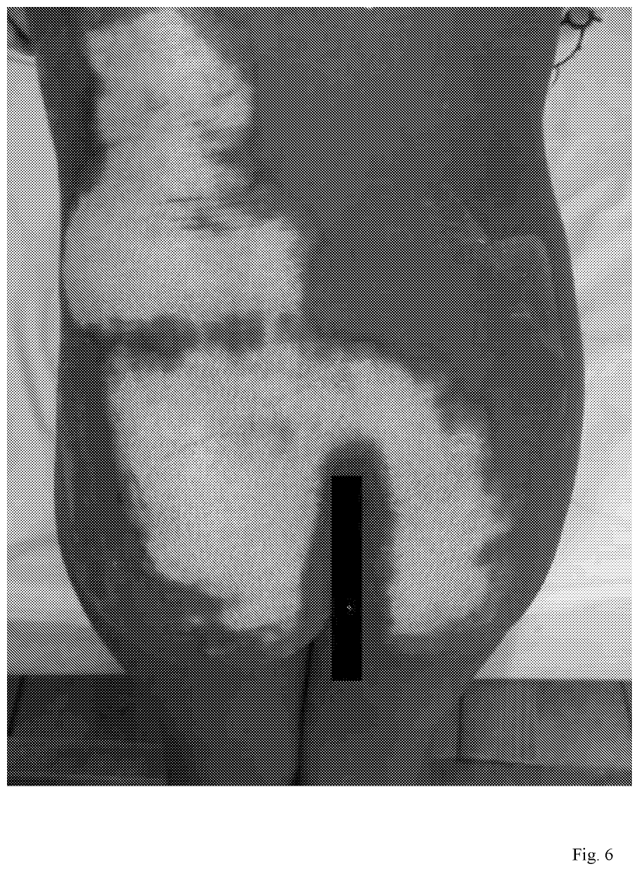

FIG. 6 there is shown part of the back and leg of a young woman who suffered from boiling water passing over her back and leg;

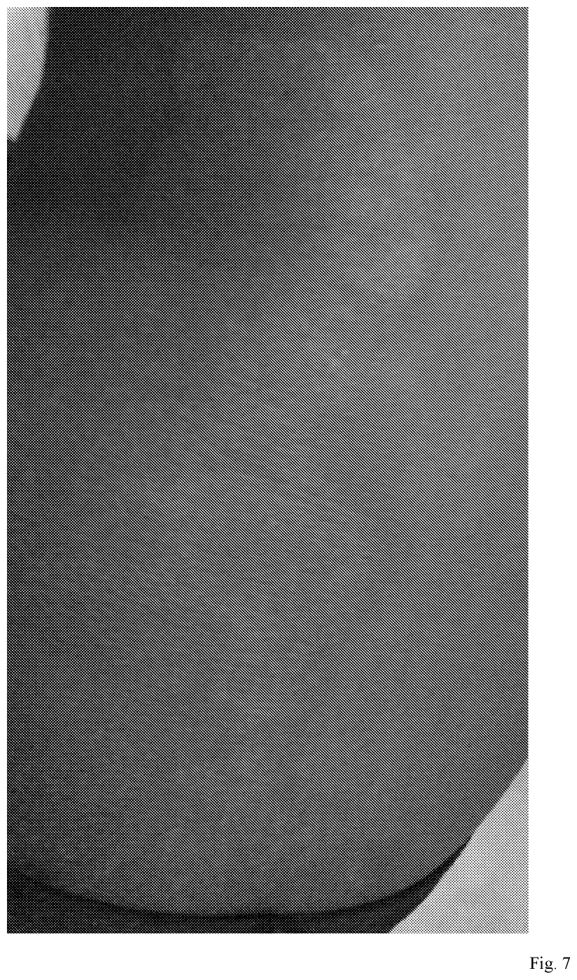

FIG. 7 shows the back of the young woman after approximately two months of the medicament of the current invention being used topically;

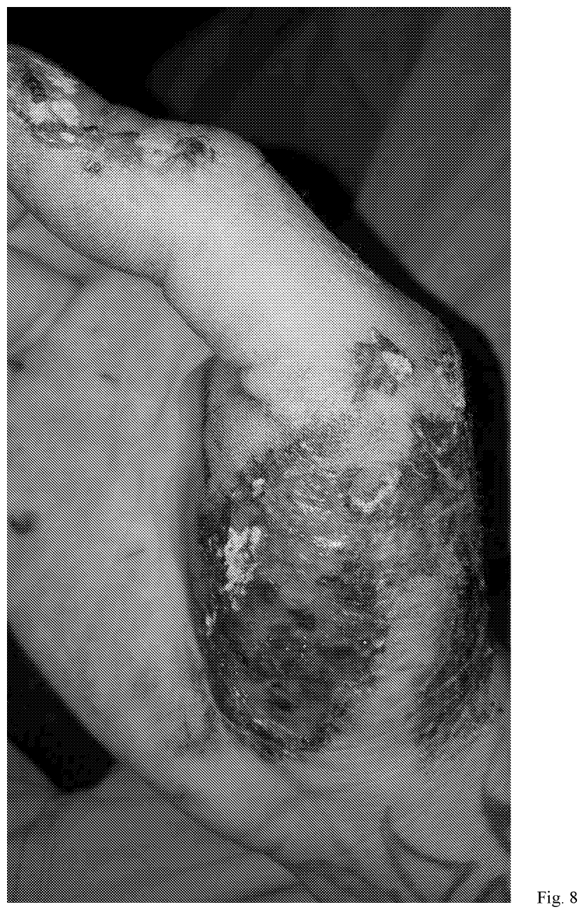

FIG. 8 shows the skin on a hand which was badly torn and abraded in a motor cycle accident;

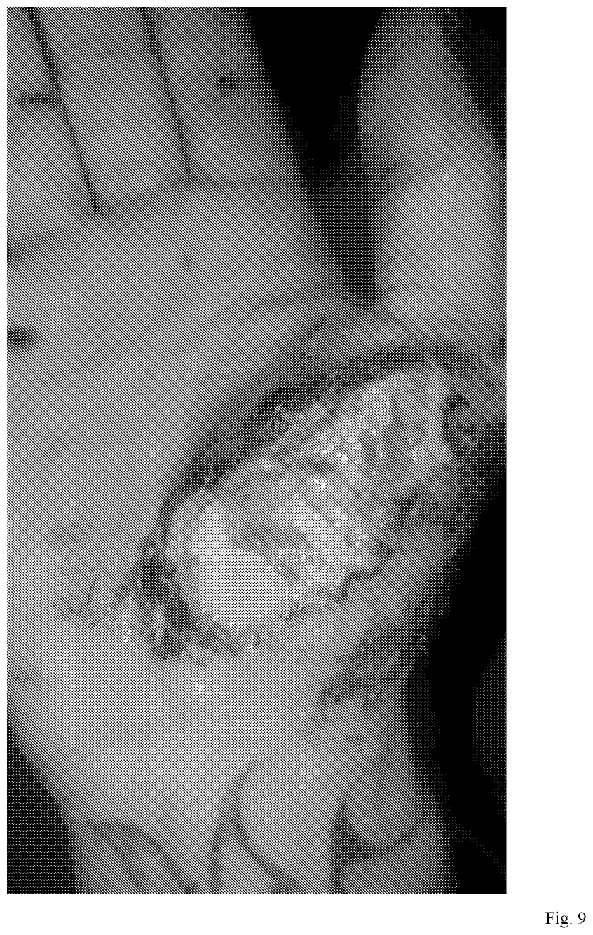

FIG. 9 shows the skin on a hand which was badly torn and abraded in a motor cycle accident after the medicament of the present invention was used to treat the abrasion; and

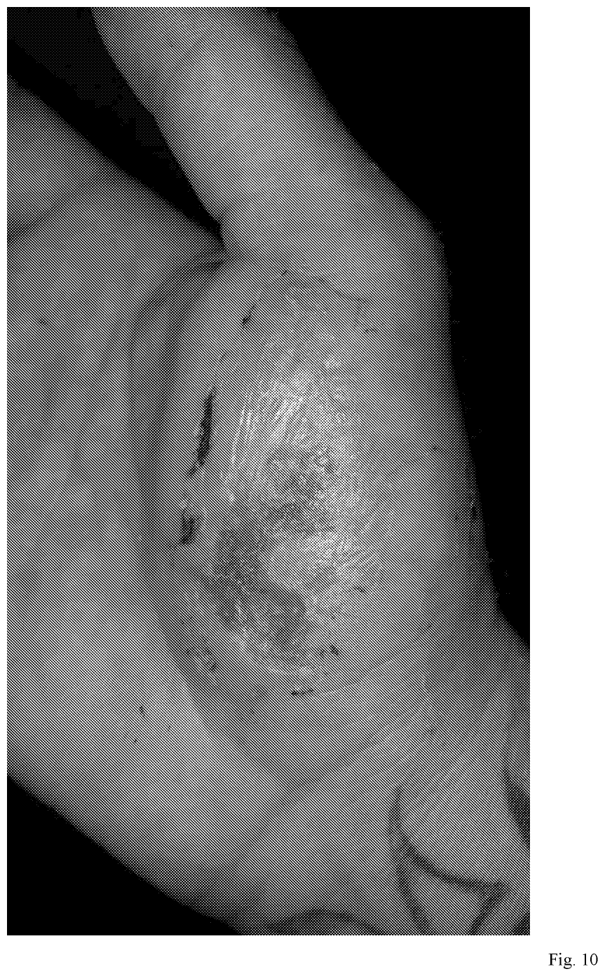

FIG. 10 shows the almost healed skin on a hand which was badly torn and abraded in a motor cycle accident after the medicament of the present invention was used to treat the abrasion;

The medicament of the invention comprises pearl infused jelly. The pearl infused jelly comprises sea pearl powder which is oxygenated for an hour with the use of an ozone generator, by which time the salt water pearl powder is saturated with oxygen. This pearl powder is mixed with liquidised petroleum jelly heated to the point where the petroleum jelly becomes liquid (between 60° C. to 70° C.). About 1% of the oxygenated salt water pearl powder is spread over the petroleum jelly and is mixed therewith. When the colour of the liquidised petroleum jelly changes from clear to a uniform opaque consistency, the petroleum jelly is at that point saturated with salt water pearl powder and the pearl powder infused jelly is allowed to cool.

The pearl infused jelly is spread thickly over sterile gauze to be applied to the wound or the pearl infused petroleum jelly is applied directly on to the wound forming a layer of pearl infused petroleum jelly which acts a protective layer keeping the wound, burn, cut, tear or abrasion clear of pollutants from the atmosphere as well as having the oxygenated saltwater pearl powder which assists in skin healing and regeneration.

The salt water pearl powder which was ground to ultrafine size preferably comprises 88 to 93% pure calcium and 7% to 12% trace elements. In more detail, the Pearl powder comprises:—selenium 1.2 mg/g; germanium 0.1 mg/g; Zinc 9.3 mg/g; Iron 2.0 mg/g; copper 0.3 mg/g; magnesium 11.3 mg/g; calcium 89-92 mg/g; trace elements 1-3 mg/g and also protein 4.5-8.3 mg/g comprising aspartic acid; serine; Arginine; Threonine; Alanine; Methionine; Isoleucine; Phenylalanine; Lysine and Glutamic acid.

Referring now to FIG. 1 there is shown a man who had burnt his face during a cooking oil accident in a commercial kitchen. The man applied the pearl infused petroleum jelly a day after the incident. The pearl infused petroleum jelly was applied at least twice daily. After a week the skin on his face had heeled significantly as can be seen in FIG. 2. After slightly more than two months his face showed very little effect of the burn, the skin had healed virtually uniformly and the pigment had regrown as can be seen in FIG. 3. FIG. 4 is a view from the side of his neck and a portion of his face a week after the accident. FIG. 5 is a picture from a similar angle, where it can be seen how well the skin had healed and how virtually all the pigment had returned. The reason the pigment had not returned as well near the ear may be due to insufficient application of the pearl infused petroleum jelly on that specific area.

Referring now to FIG. 6 there is shown part of the back and leg of a young black girl who suffered from boiling water passing over her back and leg. She suffered third-degree burns over about ½ m² of skin. The pigment under the burn disappeared. She was immediately treated with pearl infused petroleum jelly as follows:—
50 g of pearl infused petroleum jelly spread thickly over sterile gauze and was applied to the burn. This was replaced a number of times. The burn commenced healing immediately and the pain ended a few minutes after the first amount of pearl infused petroleum jelly was applied. The pearl infused petroleum jelly was reapplied daily until the burn was virtually completely healed. Furthermore, the pigment was regrown so to be difficult after two months to see where they had been burnt (as is apparent from FIG. 7).

A young man fell off a motorcycle on to a rough tar road. The skin on his hand, which was not protected by a glove, was badly torn and abraded as is shown in FIG. 8. He was taken to a place where the wound was cleaned and pearl infused petroleum jelly on sterile gauze was then applied to the wound. A week thereafter his skin was healing as can be seen in FIG. 8. The gauze infused with pearl infused petroleum jelly was removed and replaced by a fresh gauze infused with pearl infused petroleum jelly and this was repeated at least once a day every day for about two weeks until the wound started healing as shown in FIG. 9. The new healed skin on the hand is shown in FIG. 10.

In a third example a man was carrying an urn of boiling water while descending a staircase. He slipped and part of the boiling water fell to his lower calf, ankle and shoe badly burning his ankle and foot. He removed the trousers and shoe and sock. He cleaned the wound with hydrogen peroxide and then applied pearl infused petroleum jelly to the burn. Within minutes the pain was diminished and shortly after the pain had disappeared completely. The pearl infused petroleum jelly was reapplied at least once every day for two weeks when the wound was completely repaired.

In a fourth example a six-year-old boy put his hand on a stove plate. A blister formed. He then had pearl infused petroleum jelly applied to the blister which was cured in a week. Under the blister was newly formed skin.

In a fifth example a young lady was in a motor accident. The skin on her knee was ripped open. After the wound was stitched together problems arose with the wound. The medical prognosis was that the leg must be amputated. The wound was cleaned with hydrogen peroxide and then treated with Pearl infused petroleum jelly and was cleaned and treated every day for a month when the wound was healed.

In use the pearl infused petroleum jelly is applied either directly on to the area affected by the burns, skin tears, abrasions and or cuts. The pearl infused petroleum jelly may alternatively be placed on sterile gauze.

The pearl infused petroleum jelly may be used on cuts post-surgery or stiches to assist in healing with reduced scarring.

Pearl powder has proved to be as safe natural and effective medicament for burns, skin tears, abrasions and cuts.

The invention is not limited to the precise structural details hereinbefore described. For example, the pearl powder need not be oxidized before being mix with the petroleum jelly. Further the petroleum jelly may be replaced with other suitable carriers or bases, such as an oil-based cream for treating sun burn which may be further infused with vitamin E.

The petroleum jelly may be replaced with an oil-based cream. In to which the oil-based pearl powder, preferably oxygenated sea pearl powder is mixed. Although it has been found that the pearl powder separates from water-based creams, the additional of an oil into the water-based cream may be an alternative to petroleum jelly.

Further instead of a petroleum jelly the base may comprise one or more from the group of vitamin E, aloe vera, cetearyl alcohol, glycerin, phenoxyethanol, sodium hydroxide, cocoa butter, hydrogenated coconut oil, panthenol, triethanolamine, allantoin, bisabolol, butylene glycol, butyrospermum parkii butter, caprylic/capric triglyceride, dimethicone, Echinacea purpurea extract, niacinami, Chinese red oil, emu oil, castor oil or olive oil each being selected for different applications such as applying on skin that has been sunburnt, or on the body where the use of petroleum jelly is not ideal.

1. The use of pearl powder in the manufacture of a medicament for use for treating trauma.

2. The use of pearl powder in the manufacture of a medicament for use for treating trauma as claimed in claim 1 wherein the pearl powder is saltwater pearl powder.

3. The use of pearl powder in the manufacture of a medicament for use for treating trauma as claimed in claim 1 wherein the pearl powder is oxygenated saltwater pearl powder.

4. The medicament as claimed in claim 3 wherein the salt water Pearl powder is oxygenated until saturated.

5. A medicament as claimed in claim 1 wherein the pearl powder is mixed into warmed petroleum jelly.

6. A medicament as claimed in claim 2 wherein the salt water pearl powder is mixed into warmed petroleum jelly.

7. A medicament as claimed in claim 3 wherein the oxygenated pearl powder is mixed into warmed petroleum jelly.

8. A medicament as claimed in claim 2 wherein the saltwater pearl powder is mixed into an oil or oil-based cream.

9. A medicament as claimed in claim 3 wherein the oxygenated saltwater pearl powder is mixed into an oil or oil-based cream.

10. A medicament as claimed in claim 8 wherein the saltwater pearl powder is mixed into a suitable base consisting one or more items from the group of vitamin E, aloe vera, cetearyl alcohol, glycerin, phenoxyethanol, sodium hydroxide, cocoa butter, hydrogenated coconut oil, panthenol, triethanolamine, allantoin, bisabolol, butylene glycol, butyrospermum parkii butter, caprylic/capric triglyceride, dimethicone, Echinacea purpurea extract, niacinami, Chinese red oil, emu oil, castor oil or olive oil.

11. A medicament as claimed in claim 1 wherein the pearl powder is mixed into warmed petroleum jelly with the pearl powder being mixed by weight, wherein the saltwater pearl powder consists between 0.5%-5% w/w and the petroleum jelly consists 95%-99.5% w/w.

12. A medicament as claimed in claim 1 wherein the pearl powder is mixed into warmed petroleum jelly with the pearl powder being mixed by weight, wherein the saltwater pearl powder consists between 0.5%-1% w/w and the petroleum jelly consists 99%-99.5% w/w.

13. A medicament as claimed in claim 2 wherein the saltwater pearl powder is mixed into warmed petroleum jelly with the pearl powder being mixed by weight, wherein the saltwater pearl powder consists between 0.5%-5% w/w and the petroleum jelly consists 95%-99.5% w/w.

14. A medicament as claimed in claim 2 wherein the saltwater pearl powder is mixed into warmed petroleum jelly with the pearl powder being mixed by weight, wherein the saltwater pearl powder consists between 0.5%-1% w/w and the petroleum jelly consists 99%-99.5% w/w.

15. A medicament as claimed in claim 3 wherein the oxygenated saltwater pearl powder is mixed into warmed petroleum jelly with the pearl powder being mixed by weight, wherein the saltwater pearl powder consists between 0.5%-5% w/w and the petroleum jelly consists 95%-99.5% w/w.

16. A medicament as claimed in claim 3 wherein the oxygenated saltwater pearl powder is mixed into warmed petroleum jelly with the pearl powder being mixed by weight, wherein the saltwater pearl powder consists between 0.5%-1% w/w and the petroleum jelly consists 99%-99.5% w/w.

17. The medicament as claimed in claim 1 wherein the petroleum jelly with which the salt water pearl powder has been mixed is applied to sterile gauze.

18. A medicament as claimed in claim 8 wherein the saltwater pearl powder is mixed into an oil or oil-based cream with the pearl powder being mixed by weight wherein the saltwater pearl powder consists between 0.5%-5% w/w and the oil consists 95%-99.5% w/w.

19. A medicament as claimed in claim 8 wherein the saltwater pearl powder is mixed into an oil or oil-based cream with the pearl powder being mixed by weight wherein the saltwater pearl powder consists between 0.5%-1% w/w and the oil consists 99%-99.5% w/w.

20. The use of saltwater pearl powder mixed in to a petroleum jelly in the treatment of trauma.
